(19) United States
(12) Patent Application Publication
(10) Pub. No.: US 2010/0028869 A1
(43) Pub. Date: Feb. 4, 2010

(54) METHOD TO ASSESS SUSCEPTIBILITY TO ANDROGENIC ALOPECIA

(75) Inventors: Nicola PIRASTU, Pula (IT); Mario PIRASTU, Pula (IT); Dionigi Antonio PRODI, Pula (IT)

Correspondence Address: Kenneth K. Sharples;LAW OFFICE OF KENNETH K. SHARPLES, SENA PLAZA BUILDING, SUITE 54, 125 EAST PALACE AVE, SANTA FE, NM, 87501, US

(73) Assignee: SHARDNA S.P.A., Pula - Cagliari (IT)

(21) Appl. No.: 12/184,126

(22) Filed: Jul. 31, 2008

(51) Int. Cl. C12Q 1/68

(52) U.S. Cl. 435/6

(57) ABSTRACT

The present invention relates to a method to assess the susceptibility to androgenetic alopecia comprising detecting the presence of polymorphisms in the EDA2R gene.

### FIELD OF INVENTION

The present invention relates to a method to assess the susceptibility to androgenetic alopecia comprising detecting the presence of polymorphisms in the EDA2R gene.

### BACKGROUND OF INVENTION

Androgenetic alopecia (AGA) is characterized by hair loss that affects up to 50% of all males (Hamilton, 1951). Although it is generally accepted that it is a polygenic heritable trait, a clearly responsible gene has yet to be identified (Kuster and Happle, 1984; Ellis et al., 1998). The authors of the present invention carried out an epidemiological survey for AGA in eight villages of Ogliastra, a secluded area of central Sardinia (Angius et al., 2001). The population of each village is characterized by high endogamy, little immigration, slow population growth and there have been few marriage exchanges among the villages during the centuries. This was proven both by genealogical reconstructions and through genetic studies, that is, of mitochondrial DNA. For this reason, each village can be considered independently of each others (Fraumene et al., 2003).

### SUMMARY OF INVENTION

Therefore the present invention relates to a method to assess the susceptibility to androgenetic alopecia in a subject comprising detecting the presence of a single nucleotide polymorphism in the EDA2R gene, wherein the presence of said single nucleotide polymorphism is detected by genotyping the DNA from a biological sample of the subject.

In a preferred aspect the single nucleotide polymorphism causes the substitution of arginine with lysine on the amino acid 57 of SEQ ID No. 1, more preferably the single nucleotide polymorphism that causes the substitution of arginine with lysine on the amino acid 57 of SEQ ID No. 1 is rs1385699.

In population genetics, linkage disequilibrium is the non-random association of alleles at two or more loci, not necessarily on the same chromosome. It is not the same as linkage, which describes the association of two or more loci on a chromosome with limited recombination between them. Linkage disequilibrium describes a situation in which some combinations of alleles or genetic markers occur more or less frequently in a population than would be expected from a random formation of haplotypes from alleles based on their frequencies. Non-random associations between polymorphisms at different loci are measured by the degree of linkage disequilibrium (LD) (www.wikipedia.com).

In an alternative preferred aspect the single nucleotide polymorphism is in linkage disequilibrium with rs1385699, more preferably the single nucleotide polymorphism that is in linkage disequilibrium with rs1385699 is selected from the group of: rs6152, rs12558842, rs4827380, rs12855916, rs11093958 or rs1485682.

The biological sample may be selected from: blood, saliva, buccal swab.

The invention will be now described by means of non limiting examples referring to the following figures:

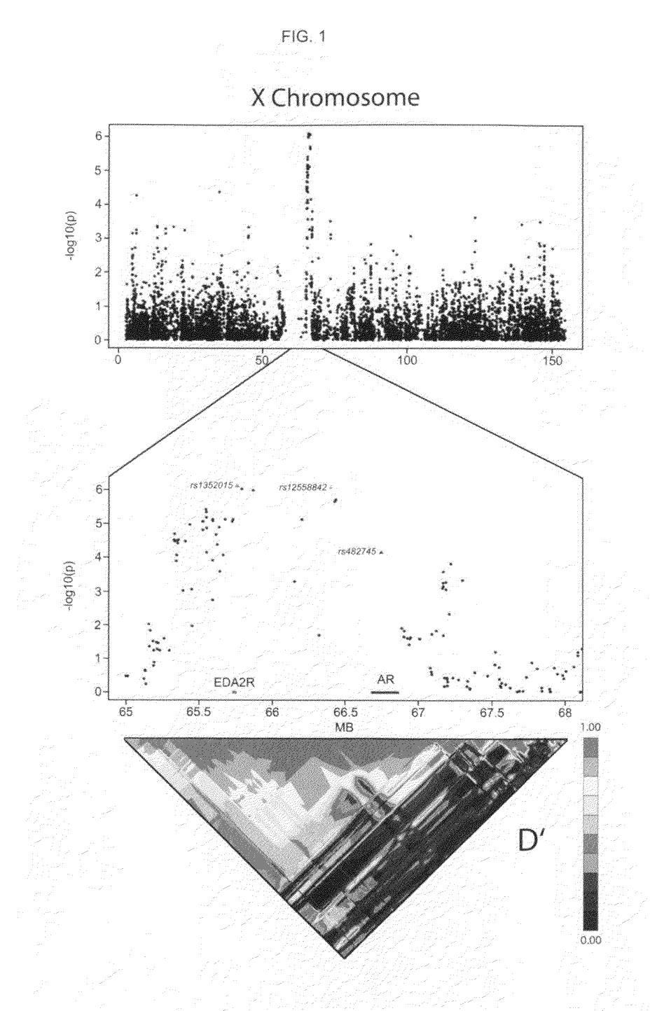

FIG. 1. Association results on X chromosome. The upper figure shows the log 10(p) for all the SNPs tested on the whole chromosome. The lower figure shows enlargement of the most significant region. The linkage disequilibrium (LD, D0) plot is shown below. The solid bars represent the genes.

### EXAMPLE 1

### Genotype on Affymetrix-GeneChip Human Mapping 500 k Array

To calculate AGA prevalence, the authors selected males older than 18 years and with a grade higher than IIv on the Norwood-Hamilton hair-loss scale. The authors found that the average mean prevalence in men was 47%, varying from 39% in the village of Seui to 56% in Talana. Given the large number of collected samples (9,000), it was possible to select the most severe cases of AGA. The authors picked men who had a baldness grade of at least IV on the Norwood-Hamilton scale and had onset before 30 years of age. For controls, the authors selected men who were at least 40 years old at the time of the visit and had no evidence of AGA. Using these parameters, the authors selected 200 cases and 200 controls; samples were selected in equal number (25 cases and 25 controls) from each of the eight villages to avoid population stratification. Within each village, the authors selected cases and controls who were the most distantly related possible (i.e., with the lowest kinship), and so that cases did not have greater kinship than controls (averaged mean kinship in cases=0.0016, SD=0.01; average mean kinship in controls=0.0011, SD=0.01). The algorithm used, involving sampling from the many pairwise relationships present in extended genealogies, is described by Falchi et al. (2004). The authors genotyped these samples with the Affymetrix-GeneChip human mapping 500 k array. The minimum call rate for each individual was 93%; for each single nucleotide polymorphism (SNP) it was 90%. All individuals participating in the study signed informed consent forms, and all the samples were taken in accordance with the Declaration of Helsinki Principles (http://www.wma.net/e/policy/17-c_e.html). The authors decided to investigate the association first on the X chromosome, not only because previous studies have associated this chromosome to AGA and but also because, on a first genome-wide scan, the most positive signal was on this chromosome. The authors tested 7,093 SNPs on the whole X chromosome using CQLS (Bourgain et al., 2003), a software that permits the correction of the association analysis results by the kinship matrix. The authors used this method because the population is inbred, and, although samples were selected to avoid spurious association due to kinship, the authors cannot exclude cryptic relatedness not accounted for. The authors used markers with minor allele frequency (MAF) higher than 0.025 in the whole sample because the authors did not have enough statistical power to detect association with rarer variants.

Genomic DNA was extracted from 7 ml of EDTA-treated blood with the guanidine isothiocianate method, as described by Ciulla (Ciulla T A, Sklar R M, Hauser S L. A simple method for DNA purification from peripheral blood. Anal Bioch 1988; 174: 485-488.)

The sample was genotyped with the Affymetrix-GeneChip® human mapping 500 k array, the minimum call rate for each individual was 93%, while the minimum call rate for each SNP was 90%.

The authors found that several markers in the Xq11-q12 region (FIG. 1, Table 1) were strongly associated with AGA.

TABLE 1
Association results of the SNPs in the EDA2R/AR region in
200 cases and 200 controls.

| SNP | Position | p-value |
|---|---|---|
| rs582787 | 65188405 | 0.057711 |
| rs670546 | 65188841 | 0.162102 |
| rs5918974 | 65191060 | 0.130534 |
| rs11093886 | 65206517 | 0.034112 |
| rs5918988 | 65212012 | 0.054128 |
| rs5918991 | 65218525 | 0.03625 |
| rs5918995 | 65228432 | 0.058916 |
| rs3940009 | 65259323 | 0.025659 |
| rs1997707 | 65293453 | 0.05825 |
| rs6624875 | 65325170 | 2.96E−05 |
| rs5964500 | 65331342 | 3.32E−05 |
| rs2206203 | 65331899 | 2.03E−05 |
| rs1264212 | 65341266 | 0.000128 |
| rs806607 | 65343765 | 8.70E−05 |
| rs806608 | 65345254 | 3.12E−05 |
| rs806610 | 65350264 | 3.74E−05 |
| rs1091486 | 65356189 | 2.96E−05 |
| rs708969 | 65386620 | 0.000981 |
| rs1456806 | 65402156 | 3.34E−05 |
| rs5919043 | 65433624 | 1.07E−05 |
| rs601552 | 65448584 | 0.00088 |
| rs1463435 | 65448903 | 0.011258 |
| rs5964522 | 65521580 | 8.47E−06 |
| rs1379146 | 65523086 | 8.47E−06 |
| rs2840240 | 65523281 | 1.53E−05 |
| rs4573425 | 65544491 | 3.88E−06 |
| rs4240047 | 65545588 | 4.48E−06 |
| rs7888975 | 65546997 | 6.48E−06 |
| rs5965192 | 65549504 | 1.36E−05 |
| rs6525067 | 65549800 | 7.01E−05 |
| rs4240049 | 65589063 | 8.36E−06 |
| rs5919108 | 65589137 | 0.000124 |
| rs4357442 | 65589319 | 7.32E−06 |
| rs5919109 | 65589338 | 0.001812 |
| rs5965213 | 65612768 | 2.15E−05 |
| rs5919126 | 65622499 | 4.21E−05 |
| rs5919135 | 65635063 | 1.28E−05 |
| rs1331101 | 65640583 | 0.000262 |
| rs5965240 | 65663804 | 8.56E−05 |
| rs6624219 | 65676567 | 7.32E−06 |
| rs1586315 | 65725320 | 8.47E−06 |
| rs5919160 | 65731297 | 7.32E−06 |
| rs1352015 | 65760568 | 7.77E−07 |
| rs1756784 | 65789770 | 9.61E−07 |
| rs16990143 | 65869018 | 1.04E−06 |
| rs471205 | 66155042 | 0.000509 |
| rs2136931 | 66205755 | 7.47E−06 |
| rs34191540 | 66321974 | 0.02066 |
| rs12558842 | 66398525 | 8.38E−07 |
| rs6625155 | 66398618 | 8.58E−07 |
| rs6625163 | 66427709 | 2.25E−06 |
| rs5965383 | 66436233 | 1.90E−06 |
| rs4827545 | 66745110 | 6.92E−05 |
| rs1415271 | 66884958 | 0.012651 |
| rs3927643 | 66898321 | 0.022861 |
| rs6625208 | 66900243 | 0.015185 |
| rs5919427 | 66920309 | 0.025129 |
| rs4370673 | 66934751 | 0.038215 |
| rs5919432 | 66938275 | 0.026187 |
| rs4456006 | 66944947 | 0.023504 |
| rs7885198 | 67005788 | 0.025903 |
| rs11094044 | 67006471 | 0.026435 |
| rs1115361 | 67078127 | 0.188392 |
| rs2363785 | 67088023 | 0.25016 |
| rs16990427 | 67090515 | 0.018996 |
| rs17302236 | 67090756 | 0.298564 |
| rs12009526 | 67119659 | 0.01518 |
| rs2768576 | 67162282 | 0.000797 |
| rs2031751 | 67165559 | 0.00057 |
| rs1327483 | 67167655 | 0.000707 |
| rs12008699 | 67168138 | 0.020926 |
| rs1327482 | 67168210 | 0.000267 |
| rs2768571 | 67175614 | 0.65635 |
| rs2765950 | 67176649 | 0.375264 |
| rs2182721 | 67184564 | 0.000895 |
| rs2768572 | 67184742 | 0.000539 |
| rs509275 | 67193148 | 0.371115 |
| rs508118 | 67194372 | 0.401107 |
| rs1410127 | 67197106 | 0.626487 |
| rs3788855 | 67198193 | 0.482599 |
| rs17217221 | 67210058 | 0.004773 |
| rs10856063 | 67218668 | 0.000151 |
| rs5965496 | 67235819 | 0.36493 |
| rs5919529 | 67274933 | 0.428604 |
| rs12389669 | 67298626 | 0.000462 |
| rs1191947 | 67329162 | 0.678295 |
| rs16990540 | 67334307 | 0.460442 |
| rs2225124 | 67348539 | 0.813401 |
| rs3788859 | 67348646 | 0.753705 |
| rs7064841 | 67383699 | 0.257581 |
| rs5965550 | 67471776 | 0.320105 |
| rs4316283 | 67520506 | 0.153635 |
| rs7357990 | 67520856 | 0.153635 |
| rs7065212 | 67522844 | 0.063969 |
| rs5919554 | 67549966 | 0.18341 |
| rs5964670 | 67550826 | 0.413205 |
| rs6625316 | 67556726 | 0.630089 |
| rs5919559 | 67561732 | 0.704195 |
| rs5919562 | 67567078 | 0.533922 |
| rs5965589 | 67593978 | 0.577758 |
| rs5919577 | 67624322 | 0.71523 |
| rs6525242 | 67671712 | 0.940443 |
| rs4827582 | 67685649 | 0.901496 |
| rs5980854 | 67711261 | 0.923231 |
| rs6525249 | 67734615 | 0.36019 |
| rs4459029 | 67773144 | 0.128968 |
| rs5936716 | 67808504 | 0.199819 |
| rs2050979 | 67840460 | 0.737669 |
| rs1935385 | 67841517 | 0.903212 |
| rs792956 | 67894575 | 0.798604 |
| rs792953 | 67898285 | 0.973132 |
| rs792952 | 67899848 | 0.973364 |
| rs5937001 | 67925789 | 0.185275 |
| rs5980736 | 67945799 | 0.287003 |
| rs241393 | 67981767 | 0.31594 |
| rs443731 | 67990048 | 0.393941 |
| rs241388 | 68006703 | 0.239104 |
| rs907150 | 68060723 | 0.174035 |
| rs1277990 | 68085631 | 0.065046 |
| rs1277992 | 68086141 | 0.08178 |
| rs5937126 | 68104346 | 0.945556 |
| rs1277958 | 68117127 | 0.050586 |
| rs17302556 | 68118992 | 0.882464 |
| rs1277962 | 68119067 | 0.039458 |
| rs1277964 | 68120563 | 0.044068 |
| rs4844160 | 68138735 | 0.121431 |
| rs2136826 | 68139858 | 0.176858 |
| rs6624381 | 68197466 | 0.660692 |
| rs5981167 | 68251184 | 0.339509 |
| rs9887052 | 68258676 | 0.696953 |
| rs5937156 | 68261039 | 0.41519 |
| rs5936657 | 68264395 | 0.61483 |
| rs4844164 | 68292819 | 0.374703 |
| rs5937173 | 68336384 | 0.345049 |
| rs5936666 | 68336708 | 0.280326 |
| rs5937174 | 68338227 | 0.381038 |
| rs5937175 | 68339202 | 0.306027 |
| rs5936668 | 68343103 | 0.330123 |
| rs2361466 | 68343374 | 0.346911 |
| rs3915920 | 68354065 | 0.06205 |
| rs5981189 | 68361095 | 0.453066 |
| rs5980804 | 68375692 | 0.64204 |
| rs5981203 | 68421213 | 0.905032 |
| rs7888054 | 68455063 | 0.074657 |
| rs10521344 | 68502567 | 0.242079 |
| rs10521345 | 68507659 | 0.580922 |
| SNP_A-4267855 | 68530863 | 0.752629 |
| rs6525299 | 68539775 | 0.26896 |
| rs5936684 | 68564093 | 0.053582 |
| rs5981241 | 68633241 | 0.670511 |
| rs6625472 | 68656267 | 0.918503 |
| rs4844364 | 68691200 | 0.984108 |
| rs943498 | 68693493 | 0.245394 |
| rs5936478 | 68723423 | 0.733589 |
| rs5936479 | 68740934 | 0.486215 |
| rs2520366 | 68845203 | 0.325623 |

All SNPs codes and sequences can be retrieved form http://www.ncbi.nlm.nih.gov/sites/entrez. In particular, rs1352015 gave the best result (P=7.77e⁻⁷). This result is still significant at the 5% level when adjusted for multiple testing using Bonferroni correction, giving a corrected P-value of 0.0144, although it does not reach genome-wide significance (genome-wide corrected P-value 0.17). The rs1352015 SNP is located 8 kb outside the 5′ end of the EDA2R (EDA-A2 receptor) gene [Annotation: Chromosome X, NC_{—}000023.9 GeneID: 60401]. This region is close to the androgen receptor gene (AR, Annotation: Chromosome X, NC_{—}000023.9, GeneID: 367), whose intragenic variants (in particular, rs6152) have been associated with the AGA phenotype (Ellis et al., 2001; Hayes et al., 2005; Hillmer et al., 2005; Levy-Nissenbaum et al., 2005). The single AR informative intragenic marker included in the Affymetrix 500K array was rs4827545, and it gave a strongly significative P-value (P=6.49e⁻⁵).

### EXAMPLE 2

### Analysis of EDA2R and AR Genes

Because the association close to the EDA2R gene was stronger than the one on the AR gene, the authors decided to investigate the possible role of these two genes in the etiology of AGA in their population. In addition, the authors also tested rs12558842 because its P-value was almost as low as that of rs1352015.

To test their results, the authors selected 492 cases (mean onset age=24 years, mean hair-loss grade=VI, mean age=54 years) and 492 controls (mean age=56 years). This new set included 127 cases and 138 controls already used in the first step of the present study. The authors initially tested the new set for two STRs already reported as being associated with AGA-polyglutamine-encoding CAG repeat and the polyglycine-encoding GGN repeat (Ellis et al., 2001)—and for rs6152 (A/G) as described by La Spada et al. (1991) and Hillmer et al. (2005).

The authors detected 16 alleles for CAG and 10 for GGN. AGA had not been associated with a particular allele but with groups of alleles on the basis of repeat number. The authors divided alleles into long and short classes on the basis of previously published findings (Ellis et al., 2001).

Case-control analysis performed on the whole sample (492 cases and 492 controls) showed a very strong association with polymorphism rs6152 (P=4.17e⁻¹²; G-allele kinship-corrected frequencies case=0.92, controls=0.76), whereas on CAG and GGN repeats, the association with AGA was weaker but still significant (P=0.01 and P=0.0004, respectively).

To study EDA2R, the authors sequenced all its exons and the 5′- and 3′-UTR (untranslated region) regions for 20 cases and 20 controls randomly chosen from the whole sample. The authors found only an informative nsSNP on exon 2: rs1385699 (C/T), which causes the substitution of arginine with lysine on amino acid 57 of the EDA2R protein (EMBL accession AL353136.21, SEQ ID No. 1):

MDCQENEYWDQWGRCVTCQRCGPGQELSKDCGYGEGGDAYCTACPPRRYK
SSWGHHRCQSCITCAVINRVQKVNCTATSNAVCGDCLPRFYRKTRIGGLQ
DQECIPCTKQTPTSEVQCAFQLSLVEADTPTVPPQEATLVALVSSLLVVF
TLAFLGLFFLYCKQFFNRHCQRGGLLQFEADKTAKEESLFPVPPSKETSA
ESQVSENIFQTQPLNPILEDDCSSTSGFPTQESFTMASCTSESHSHWVHS
PIECTELDLQKFSSSASYTGAETLGGNTVESTGDRLELNVPFEVPSP.

Four additional informative SNPs in the 5′-UTR (rs4827380, rs12855916, rs11093958 and rs1485682) that were all in complete LD with rs1385699 in all 40 samples were also found. Genotypes for this SNP were obtained using Applied Biosystems TaqMan SNP Genotyping Assays.

rs1385699 was revealed to have the strongest association, with a P-value of 3.9e⁻¹⁹ (T-allele kinship-corrected frequencies cases=0.92, controls=0.7), odds ratio 4.65 (95% confidence intervals; 3.15-6.87). rs12558842 resulted in strong association with AGA (P-value 7.6e⁻¹⁴); it is, however, in very strong LD with rs1385699. The role of the amino acid in position 57 in EDA2R protein is not defined, but it is located in a cysteine-rich domain (Yan et al., 2000).

Both amino acids have a polar basic chain, but the N terminal group on Arg is bigger and is more basic and could influence the protein's activity. Two receptors for EDA were found that are specific for the two isoforms EDA-A1 and EDAA2: EDAR and EDA2R, respectively.

EDA-A1 and its receptor EDAR are capable of activating the NF-kB pathway and are implicated in hair growth (Botchkarev and Fessing, 2005).

EDA2R is capable of activating the NF-kB pathway and also through TRAF3,6, JNK (c-Jun N-terminal kinase) (Sinha et al., 2002), which activates c-Jun. Mutations in EDA and EDAR give rise to ectodermal dysplasia, a clinical syndrome characterized by loss of hair, sweat glands, and teeth, whereas mutations in EDA2R do not (Monreal et al., 1999; Naito et al., 2002; Newton et al., 2004). Recently, a preliminary report suggested that EDAR may influence hair thickness in Asians (A. Fujimoto, R. Kimura, J. Ohashi, U. Samakkarn, W. Settheetham-Ishida, T. Ishida, Y. Morishita, T. Furusawa, M. Nakazawa, R. Ohtsuka, R. Yuliwulandari, L. Batubara, M. S. Mustofa, K. Tokunaga, A scan for genetic determinants of human hair morphology: EDAR is associated with Asian hair thickness, ASHJ Meeting 2007). EDA2R could influence the onset of AGA through the activation of the NFkB pathway or by c-Jun, which has been shown to be critical for AR trans-activation (Bubulya et al., 1996). Moreover, in adult mice, EDA2R is also expressed in the hair bulb and in differentiating hair matrix (Botchkarev and Fessing, 2005).

Looking at the human expression data from the UniGene database (http://www.ncbi.nlm.nih.gov/sites/entrez), the authors noticed that it is expressed during embryonic life and, especially, in the first weeks after birth. Expression then seems to be absent until the 17th year of age, when it recurs in different tissues, including skin. This expression pattern fits very well with the course of AGA, with its onset around puberty.

The present study shows that AR and EDA2R are significantly associated with AGA. However, there is some LD between the two most associated markers for each gene (rs6152, rs1385699: D0=0.74, r2=0.43). To test if they are independently associated, the authors conditioned the analysis of each gene to the other one. The authors used the UNPHASED software (Dudbridge, 2003), which permits the association of a marker to be conditioned to the presence of another marker. The analysis of rs1385699 conditioned to the presence of rs6152 gave a very significant P-value of 6.136e⁻⁹, whereas when the authors conditioned the analysis of rs6152 to the presence of rs1385699 the P-value was 0.04. Again, rs1385699 conditioned to the presence of rs12558842 gave a very significant result (P-value 0.007), whereas rs12558842 conditioned to the presence of the EDA2R variant did not give a significant result (P-value 0.06).

These results show that in the studied population, the EDA2R gene variation causes susceptibility to AGA. The conditioned analysis suggests that markers on the AR gene could be associated because of LD. However, the authors cannot exclude that other variants in LD with both genes (that is, regulatory elements of either or both genes) could be associated with AGA. Moreover, the functional importance of AR has already been proven by many means, and its involvement in this pathology cannot be excluded.

### REFERENCES

Angius A, et al. (2001) Hum Genet 109:198-209
Botchkarev V A, Fessing M Y (2005) J Investig Dermatol Symp Proc 10:247-51
Bourgain C, et al. (2003) Am J Hum Genet 73:612-26
Bubulya A, Wise S C, Shen X Q, Burmeister L A, Shemshedini L (1996) J Biol Chem 271:24583-9
Dudbridge F (2003) Genet Epidemiol 25:115-21
Ellis J A, Scurrah K J, Cobb J E, Zaloumis S G, Duncan A E, Harrap S B (2007) Hum Genet 121:451-7
Ellis J A, Stebbing M, Harrap S B (1998) J Invest Dermatol 110:849-53
Ellis J A, Stebbing M, Harrap S B (2001) J Invest Dermatol 116:452-5
Falchi M, et al. (2004) Am J Hum Genet 75:1015-31
Fraumene C, Petretto E, Angius A, Pirastu M (2003) Hum Genet 114:1-10
Hamilton J B (1951) Ann N Y Acad Sci 53:708-28
Hayes V M, Severi G, Eggleton S A, Padilla E J, Southey M C, Sutherland R L et al. (2005) Cancer Epidemiol Biomarkers Prev 14:993-6
Hillmer A M, et al. (2005) Am J Hum Genet 77:140-8
Kuster W, Happle R (1984) J Am Acad Dermatol 11:921-6
La Spada A R, Wilson E M, Lubahn D B, Harding A E, Fischbeck K H (1991) Nature 352:77-9
Levy-Nissenbaum E, Bar-Natan M, Frydman M, Pras E (2005) Eur J Dermatol 15:339-40
Monreal A W, et al. (1999) Nat Genet 22:366-9
Naito A, et al. (2002) Proc Natl Acad Sci USA 99:8766-71
Newton K, French D M, Yan M, Frantz G D, Dixit V M (2004) Mol Cell Biol 24:1608-13
Sinha S K, Zachariah S, Quinones H I, Shindo M, Chaudhary P M (2002) J Biol Chem 277:44953-61
Yan M, et al. (2000) Science 290:523-52

1. A method to assess the susceptibility to androgenetic alopecia in a subject comprising detecting the presence of a single nucleotide polymorphism in the EDA2R gene, wherein the presence of said single nucleotide polymorphism is detected by genotyping the DNA from a biological sample of the subject.

2. The method of claim 1 wherein the single nucleotide polymorphism causes the substitution of arginine with lysine on the amino acid 57 of SEQ ID No. 1.

3. The method of claim 2 wherein the single nucleotide polymorphism that causes the substitution of arginine with lysine on the amino acid 57 of SEQ ID No. 1 is rs1385699.

4. The method of claim 1 wherein the single nucleotide polymorphism is in linkage disequilibrium with rs1385699.

5. The method of claim 4 wherein the single nucleotide polymorphism that is in linkage disequilibrium with rs1385699 is selected from the group of: rs6152, rs12558842, rs4827380, rs12855916, rs11093958 or rs1485682.

6. The method of claim 6 wherein the biological sample is selected from: blood, saliva, buccal swab.


SEQUENCE LISTING (1 sequence)

SEQ ID NO: 1 - protein, 297 aa, Homo sapiens
MDCQENEYWDQWGRCVTCQRCGPGQELSKDCGYGEGGDAYCTACPPRRYKSSWGHHRCQSCITCAVINRVQKVNCTATSNAVCGDCLPRFYRKTRIGGLQDQECIPCTKQTPTSEVQCAFQLSLVEADTPTVPPQEATLVALVSSLLVVFTLAFLGLFFLYCKQFFNRHCQRGGLLQFEADKTAKEESLFPVPPSKETSAESQVSENIFQTQPLNPILEDDCSSTSGFPTQESFTMASCTSESHSHWVHSPIECTELDLQKFSSSASYTGAETLGGNTVESTGDRLELNVPFEVPSP